(19) United States
(12) Patent Application Publication
(10) Pub. No.: US 2011/0004063 A1
(43) Pub. Date: Jan. 6, 2011

(54) BALLOON CONTROL APPARATUS AND CONTROL METHOD THEREOF

(76) Inventors: Takayuki NAKAMURA, Ashigarakami-gun (JP); Tsuyoshi Ashida, Ashigarakami-gun (JP); Shinichi Yamakawa, Ashigarakami-gun (JP)

Correspondence Address: BIRCH STEWART KOLASCH & BIRCH, PO BOX 747, FALLS CHURCH, VA, 22040-0747, US

(21) Appl. No.: 12/828,902

(22) Filed: Jul. 1, 2010

(30) Foreign Application Priority Data
Jul. 3, 2009 (JP) 2009-158652

(51) Int. Cl. A61B 1/01

(52) U.S. Cl. 600/115

(57) ABSTRACT

A balloon control apparatus, comprising:
an insertion part to be inserted into a luminal part;
a balloon provided at a distal end of the insertion part;
a pipeline connected to the balloon;
a pump that flows a fluid to the balloon via the pipeline;
an intermittent device that causes the pump and the pipeline to be intermittently disconnected/communicated, thereby restricting the flow of the fluid;
a pipeline pressure detection device that detects a pipeline internal pressure of the pipeline;
a flow amount calculation device that calculates a flow amount of the fluid at the time of a next communication of the intermittent device based on the pipeline internal pressure at a reached value of the pipeline internal pressure in a disconnection period of the intermittent device; and
an intermittent operation control device that controls an intermittent operation in the intermittent device based on the flow amount of the fluid calculated by the flow amount calculation device.

### BACKGROUND OF THE INVENTION

1. Field of the Invention

The present invention relates to a balloon control apparatus and a control method thereof, and particularly to a balloon control apparatus characterized by the control of the inflation/deflation time of a balloon, and a control method thereof.

2. Description of the Related Art

Conventional small intestine endoscopes are likely to be superceded by a double-balloon type endoscope. The double-balloon type endoscope is made up of: a balloon type endoscope which is attached with a balloon at an endoscope distal end and is able to supply/suck air to and from the balloon; and an overtube into which an insertion part of the balloon type endoscope is inserted and which is attached with a balloon at the tube distal end and is able to supply/suck air to and from the balloon. Moreover, the double-balloon type endoscope is connected with a balloon control apparatus for separately inflating each balloon, and the balloon control apparatus separately supplies/sucks air to and from each balloon.

When the double-balloon type endoscope is inserted into a small intestine, each balloon is alternately inflated so that the endoscope distal end or the overtube distal end is alternately fixed to the intestinal wall, and the endoscope insertion part of non-fixed side or the overtube and the small intestine are relatively moved. This will cause the intestinal tract to be hauled in with a portion fixed by the balloon serving as a fulcrum, allowing the distal end of the double-balloon type endoscope to be relatively inserted into a deep part of the small intestine.

Regarding a balloon at the distal end part of the double-balloon type endoscope of this type, a method of measuring a balloon internal pressure and controlling the pressure of a pump in order to safely and rapidly inflate/deflate the balloon is disclosed (Japanese Patent Application Laid-Open No. 2007/203035).

However, the measurement method of the balloon internal pressure, in which a pressure sensor is attached in the vicinity of an air feed/discharge pump from the balloon via a long air feed/discharge pipe, cannot measure a correct internal pressure of the balloon during air feed/discharge due to the effect of a pressure loss (in accordance with the air feed/discharge flow velocity) in the air feed/discharge pipe, resulting in a relationship, “the pressure at the position of the pressure sensor=the balloon internal pressure+the pressure loss of the air feed/discharge pipe.”

On the other hand, the pressure in the vicinity of the pump, which is the pressure measurement position, becomes high because the pipeline which leads the fluid to the inflator body (balloon) is long: therefore, particularly when the pipeline is thin, a high speed feeding of fluid will result in an increase in the pressure rise due to a pipeline pressure loss. Because of this, the fluid amount of the fluid that can be fed by one intermittent operation in the control based on the pressure at the pressure sensor position decreases, and a problem arises in that it takes time for the inflator body to reach a desired pressure.

### SUMMARY OF THE INVENTION

The present invention has been made in view of such circumstances, and has its object to provide a balloon control apparatus that can substantially reduce the time for reaching a desired internal pressure of the balloon, while appropriately ensuring a pressure to act on the inner wall of the luminal part, and a control method thereof.

In order to achieve the above described object, a balloon control apparatus according to a first aspect of the present invention is configured to include: an insertion part to be inserted into a luminal part; a balloon provided at a distal end of the insertion part; a pipeline connected to the balloon; a pump that flows a fluid to the balloon via the pipeline; an intermittent device that causes the pump and the pipeline to be intermittently disconnected/communicated, thereby restricting the flow of the fluid; a pipeline pressure detection device that detects a pipeline internal pressure of the pipeline; a flow amount calculation device that calculates a flow amount of the fluid at the time of a next communication of the intermittent device based on the pipeline internal pressure at a reached value of the pipeline internal pressure in a disconnection period of the intermittent device; and an intermittent operation control device that controls an intermittent operation in the intermittent device based on the flow amount of the fluid calculated by the flow amount calculation device.

Since, in the balloon control apparatus according to the first aspect, a pipeline internal pressure of the pipeline is detected by the pipeline pressure detection device; a flow amount of the fluid at the time of a next communication of the intermittent device is calculated by the flow amount calculation device based on the pipeline internal pressure at a reached value of the pipeline internal pressure in a disconnection period of the intermittent device; and the intermittent operation in the intermittent device is controlled by the intermittent control device based on the flow amount of the fluid calculated by the flow amount calculation device, it is possible to substantially reduce the time for reaching a desired internal pressure of the balloon while appropriately ensuring a pressure to act on the inner wall of the luminal part.

As described in the balloon control apparatus according to a second aspect of the present invention, it is preferable that in the balloon control apparatus according to the first aspect, the pipeline pressure detection device detects the pipeline internal pressure of the pipeline between the balloon and the intermittent device.

As described in the balloon control apparatus according to a third aspect of the present invention, it is preferable that in the balloon control apparatus according to the first or second aspect, the flow amount calculation device includes: a target internal pressure value storage device that prestores a target internal pressure value of the balloon; a reached value detection device that detects a reached value of the pipeline internal pressure in a disconnection period of the intermittent device; and a pressure difference calculation device that calculates a pressure difference between the pipeline internal pressure at the reached value detected by the reached value detection device and the target internal pressure value stored in the target internal pressure value storage device, and calculates a flow amount of the fluid at the time of a next communication of the intermittent device based on the pressure difference calculated by the pressure difference calculation device.

As described in the balloon control apparatus according to a fourth aspect of the present invention, it is preferable that in the balloon control apparatus according to any one of the first to third aspects, the intermittent operation control device controls at least one of a communication time in the intermittent operation in the intermittent device and a flow pressure of the pump based on the flow amount of the fluid calculated by the flow amount calculation device.

As described in the balloon control apparatus according to a fifth aspect of the present invention, it is preferable that in the balloon control apparatus according to any one of the first to fourth aspects, the pump flows and supplies the fluid to the balloon.

As described in the balloon control apparatus according to a sixth aspect of the present invention, it is preferable that in the balloon control apparatus according to any one of the first to fourth aspects, the pump flows and discharges the fluid from the balloon.

As described in the balloon control apparatus according to a seventh aspect of the present invention, it is preferable that in the balloon control apparatus according to any one of the first to sixth aspects, the insertion part is an insertion part of an endoscope that is inserted into a luminal organ in a body cavity.

A method of controlling the balloon control apparatus according to an eighth aspect of the present invention is configured to include: an intermittent step of causing a pump, which flows a fluid into a balloon provided at a distal end of an insertion part to be inserted into a luminal part, and a pipeline, which is connected to the balloon, to be intermittently disconnected/communicated thereby restricting the flow of the fluid; a pipeline pressure detection step of detecting a pipeline internal pressure of the pipeline; a flow amount calculation step of calculating a flow amount of the fluid at the time of a next communication of the intermittent step based on the pipeline internal pressure at a reached value of the pipeline internal pressure in a disconnection period of the intermittent step; and an intermittent operation control step of controlling an intermittent operation in the intermittent step based on the flow amount of the fluid calculated by the flow amount calculation step.

As so far described, the present invention has an effect of minimizing the time required for a balloon to reach a desired pressure while appropriately ensuing a pressure to act on the inner wall of a luminal part.

### BRIEF DESCRIPTION OF THE DRAWINGS

FIG. 1 is a system configuration diagram of an endoscope apparatus including a balloon control apparatus for a double-balloon type endoscope;

FIG. 2 shows an example of a connector (a balloon air feed port) of the overtube side and a connector at a distal end of the tube for FIG. 1;

FIG. 3 is a block diagram to show an air supply control section of the balloon control apparatus of the FIG. 1;

FIG. 4 is a flowchart to show the flow of the control in the balloon control apparatus of FIG. 3;

FIG. 5 is an explanatory diagram to show the state of the balloon according to the processing of FIG. 4; and

FIG. 6 shows a transition of the internal pressure of the balloon according to the processing of FIG. 4.

### DETAILED DESCRIPTION OF THE PREFERRED EMBODIMENTS

Hereafter, a balloon control apparatus relating to the present invention will be described in detail with reference to appended drawings.

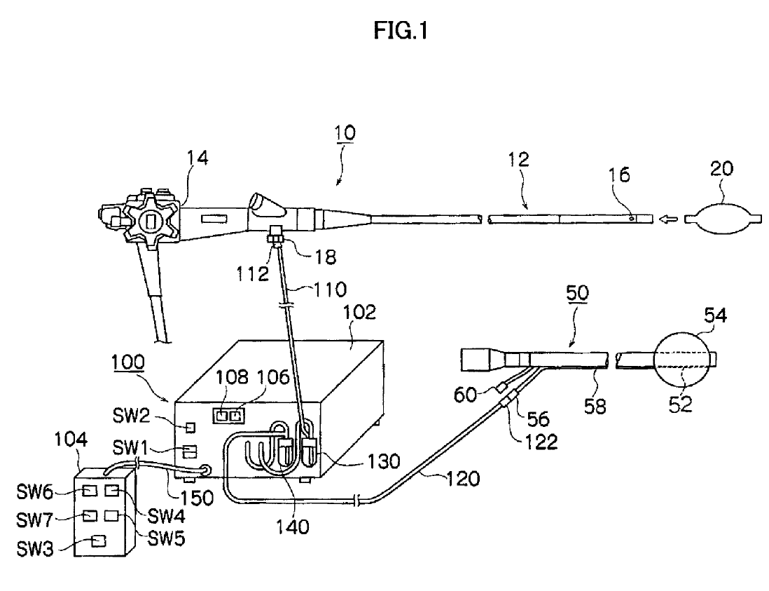

FIG. 1 is a system configuration diagram of an endoscope apparatus including a balloon control apparatus for a double-balloon type endoscope.

As shown in FIG. 1, this endoscope apparatus is made up of a double-balloon type endoscope including a balloon type endoscope 10 and an overtube 50, and a balloon control apparatus 100.

The balloon type endoscope 10 is an electronic endoscope which is provided with a photographing lens and an image pickup element (for example, CCD) etc. in the distal end of an insertion part 12, and an observed image is formed into an image on a CCD via a photographing lens, and is photoelectrically converted here. The photoelectrically converted electric signal which indicates an observed image is outputted to a processor not shown via a wiring in the insertion part 12 and a hand control part 14, and is subjected to an appropriate signal processing thereafter being outputted to a monitor TV. This allows the observed image to be displayed on the monitor TV.

Moreover, an air supply/suction port 16 is provided on a distal-end side face of the insertion part 12 of the balloon type endoscope, and on the other hand, a balloon air feed port 18 is provided on the side of the hand control part 14 so that the air supply/suction port 16 and the balloon air feed port 18 are connected by an air supply tube having an inner diameter of about 0.8 mm and provided along the insertion part 12.

When this balloon type endoscope 10 is used as a double-balloon type endoscope, the distal end of the insertion part 12 is covered with a balloon 20 and the both ends of the balloon 20 are fixed by a fixing rubber band. This allows air to be supplied to the balloon 20 from the balloon air feed port 18 via the air supply/suction port 16 causing the balloon 20 to be inflated, and air in the balloon 20 to be sucked causing the balloon to be deflated (intimate contact with the insertion part distal end).

An overtube 50, which is for the purpose of inserting the insertion part 12 of the balloon type endoscope 10 into a deep part of a small intestine in cooperation with the balloon type endoscope 10, has an inner diameter slightly larger than the outer diameter of the insertion part 12 of the balloon type endoscope 10, and a flexibility as with the insertion part 12 of the balloon type endoscope 10.

An air supply/suction port 52 is provided on the distal-end side face of the overtube 50, and a balloon 54 is attached around the tube distal end in such a way to surround the air supply/suction port 52. Moreover, a balloon air feed port 56 is provided in the rear part of the overtube 50, and the balloon air feed port 56 and the air supply/suction port 52 are connected by an air supply tube 58 having an inner diameter of about 1 mm and integrally formed along the outer periphery of the overtube 50. The above described configuration allows air to be supplied into the balloon 54 from the balloon air feed port 56 via an air supply tube 58 and an air supply/suction port 52 to inflate the balloon 54, and air in the balloon 54 to be sucked to deflate the balloon 54. It is noted that a water injection port 60 is a water injection port for injecting a lubricant (water) into the overtube 50.

A balloon control apparatus 100, which separately performs air supplying/suctioning to and from each balloon 20, 54 to alternately inflate the balloon 20 at the insertion part distal end of the balloon type endoscope 10 and the balloon 54 at the distal end of the overtube 50, is made up of an apparatus main unit 102 provided with a pump, a sequencer, etc., and a remote control hand switch 104.

A power supply switch SW1, a stop switch SW2 which is to be operated at the time of an emergency, a pressure gauge 106 for the balloon 20, a pressure gauge 108 for the balloon 54, and the like are provided in the front panel of the apparatus main unit 102 of the balloon control apparatus 100.

Moreover, tubes 110 and 120, which serve as a pipeline for the air supply/suction to and from each balloon 20, 54, are attached to the front panel of the apparatus main unit 102. It is noted that each tube 110, 120 has an inner diameter of about 6 mm.

A liquid reservoir tank 130 for the endoscope and a liquid reservoir tank 140 for the overtube, which are respectively for the purpose of preventing a reverse flow of body fluid when the balloon is broken, is provided at some midpoint of each tube 110, 120, and each liquid reservoir tank 130, 140 is detachably attached to the front panel of the apparatus main unit 102.

The tube 110 and the tube 120 are formed such that each tube has a different color and pattern, and a connector 112, 122 has a different shape, size, etc. in order to prevent connection errors such as that the tube 110 is connected to the balloon air feed port 56 of the overtube side, and the tube 120 is connected to the balloon air feed port 18 of the endoscope side.

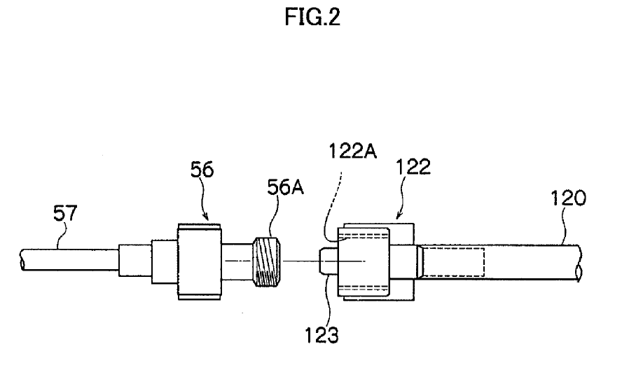

FIG. 2 shows an example of the connector of the overtube side and the connector (balloon air feed port) at the distal end of the tube of FIG. 1.

As shown in FIG. 2, a male screw 56A is formed in one end of the connector of the balloon air feed port 56, and the center of the connector of the balloon air feed port 56 including the male screw 56A is formed to be hollow, and connected with the tube 57 which is to be connected to the overtube 50.

On the other hand, the connector 122 at the distal end of the tube 120 is formed with a female screw 122A which is to be threadedly engaged with the male screw 56A of the connector of the balloon air feed port 56, and a protruding part 123 to be inserted into the male screw 56A of the connector of the balloon air feed port 56 is formed in the center of the female screw 122A. The center of the connector 122 including the protruding part 123 is formed to be hollow, and is connected with the tube 120.

The balloon air feed port 56 and the connector 122 are configured to be threadedly engaged with each other, thereby being allowed to be hermetically connected.

Here, as shown in FIG. 1, the connector 112 of the tube 110 of the endoscope side and the connector 122 (see FIG. 2) of the tube 120 are formed to have a different connector shape and a size (including the diameter and pitch of screw) from each other. This disables to perform erroneous connections (prevents connection errors) such as connecting the connector 112 to the balloon air feed port 56 of the overtube side, and connecting the connector 122 to the balloon air feed port 18 of the endoscope side.

It is noted that the connector (balloon air feed port) 18 of the endoscope side and the connector (balloon air feed port) 56 of the overtube side are also, without saying, formed to have different connector shapes and sizes, etc. so as to be connectable with corresponding connectors 112 and 122.

Further, in order to prevent connection errors between the balloon air feed port 56 provided in the overtube 50 and the water injection port 60, the balloon air feed port 56 and the water injection port 60 are formed to have different shapes and sizes, or colors and patterns of the tube, etc.

On the other hand, the hand switch 104 is provided with a stop switch SW3, which is similar to a stop switch SW2 provided at the apparatus main unit 102 side, an endoscope ON/OFF switch SW4 for instructing the compression/decompression of the balloon 20 of the endoscope side, a pause switch SW5 for maintaining the pressure of the balloon 20 of the endoscope side, an overtube ON/OFF switch SW6 for instructing the compression/decompression of the balloon 54 of the overtube side, and a pause switch SW7 for maintaining the pressure of the balloon 54 of the overtube side; and this hand switch 104 is electrically connected to the apparatus main unit 102 via a cord 150.

Next, the operation when a double-balloon type endoscope of above described configuration is used will be described.

The insertion part 12 of the balloon type endoscope 10 is inserted into the overtube 50, the tube 110 of the balloon control apparatus 100 is connected to the balloon air feed port 18 of the endoscope side, and tube 120 is connected to the balloon air feed port 56 of the overtube side.

Next, while the insertion part of the double-balloon type endoscope is inserted into a small intestine via a stomach or colon, the balloons 20 and 54 are alternately inflated when they are inserted into a deep part of the small intestine. That is, the endoscope ON/OFF switch SW4 of the hand switch 104 is turned ON to instruct a compression so that air is supplied to the balloon 20 from the apparatus main unit 102 of the balloon control apparatus 100 via a tube 110 to inflate the balloon 20 until a predetermined compressive force is reached. This allows the insertion part distal end of the balloon type endoscope 10 to be fixed to the intestinal wall. On the other hand, the overtube ON/OFF switch SW6 of the hand switch 104 is turned OFF to instruct a decompression, and air is sucked until the tube 120, etc. connected with the balloon 54 becomes a predetermined negative pressure, to deflate the balloon such that the overtube 50 and the intestinal tract are relatively movable.

In this state, the overtube 50 is advanced until the distal end of the overtube 50 reaches near the distal end of the balloon type endoscope 10.

Next, on the contrary to the above described case, the overtube ON/OFF switch SW6 of the hand switch 104 is turned ON to instruct a compression, and air is supplied from the apparatus main unit 102 to the balloon 54 via the tube 120 to inflate the balloon 54 until a predetermined compression force is reached. This allows the distal end of the overtube 50 to be fixed to the intestinal wall. On the other hand, the endoscope ON/OFF switch SW4 of the hand switch 104 is turned OFF to instruct a decompression, and air is sucked until the tube 110, etc. connected with the balloon 20 becomes a predetermined negative pressure to deflate the balloon 20 so that the insertion part 12 of the balloon type endoscope 10 and the intestinal tract are relatively movable, and the insertion part 12 of the balloon type endoscope 10 is advanced.

While repeating the above described operation and thereby moving the fixing point by each balloon further and further into a deeper part, the distal end of the double-balloon type endoscope is advanced. When a portion where a complicated loop is formed is reached, the overtube 50 is slowly pulled together with the balloon type endoscope 10 with both the balloons 20 and 54 being inflated. This operation will cause the loop to be simplified without causing the endoscope distal end to come out, so that the intestinal tract inserted with the endoscope will be contracted so as to be folded in on the overtube 50. Repeating the above described series of operation, the insertion into a deeper small intestine is performed while folding in the intestinal tract on the overtube 50 thereby simplifying the loop of the intestinal tract.

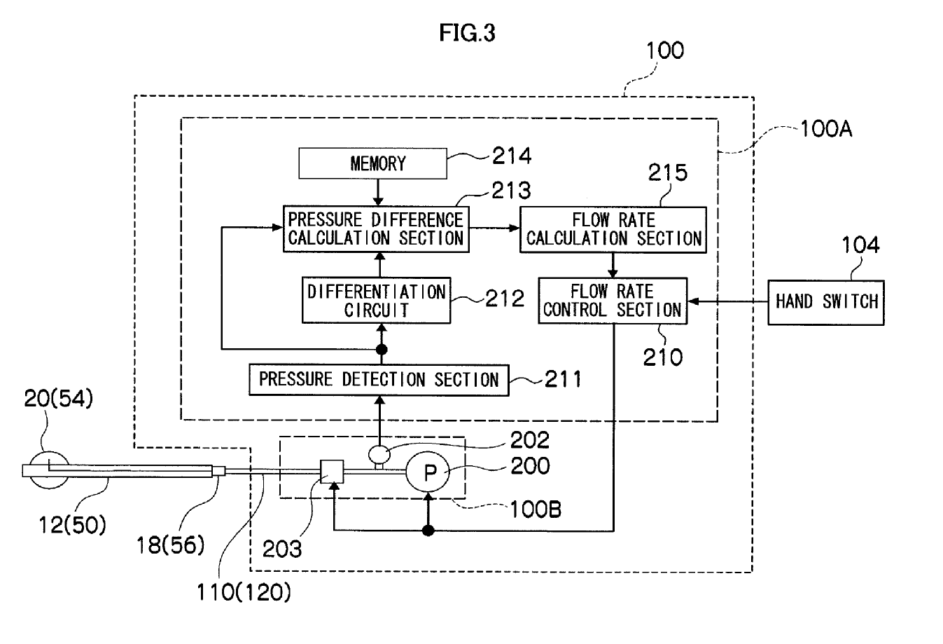

Next, the balloon control apparatus 100 will be described on its internal structure taking an example of the air supply to the balloon 20. FIG. 3 is a block diagram to show an air supply control section of the balloon control apparatus of FIG. 1. It is noted that an air discharge control section of the balloon 20, and an air supply control section and air discharge control section of the balloon 54 are configured in a similar manner.

As shown in FIG. 3, the balloon control apparatus 100 is configured such that a supply control section 100A and an air supply section 100B make up an air supply control section.

The air supply section 100B is configured to include a pump 200 connected to the tube 110, a pressure gauge 202 that measures the internal pressure of the tube 110, and an electromagnetic valve 203 as an intermittent device which is provided between the balloon air feed port 18 and the pump 200, and causes the tube 110 to be intermittently communicated/disconnected.

On the other hand, the supply control section 100A is configured to include a pressure detection section 211 as a pipeline pressure detection device, a differentiation circuit 212 as a reached value detection device, a pressure difference calculation section 213 as a pressure difference calculation device, a memory 214 as a target internal pressure value storage device, a flow rate calculation section 215, and a flow rate control section 210.

It is noted that the flow amount calculation device is made up of the above described pressure detection section 211, the differentiation circuit 212, the pressure difference calculation section 213, the memory 214, the flow rate calculation section 215, and the flow rate control section 210.

The pressure detection section 211 detects the internal pressure of the tube 110 measured by the pressure gauge 202, as a pressure signal (electric signal), and the differentiation circuit 212 differentiates the pressure signal in time to extract a reached value of the pressure signal.

The pressure difference calculation section 213 calculates a pressure difference (P−Pn) between the internal pressure (pressure signal value) Pn of the tube 110 at a reached value of the pressure signal extracted at the differentiation circuit 212 and a target internal pressure P of the balloon 20 prestored in the memory 214.

The flow rate calculation section 215 calculates a supply flow rate Vn of air at the time of a next communication of the electromagnetic valve 203 based on a predetermined function of which variable is the pressure difference (P−Pn) calculated by the pressure difference calculation section 213.

The flow rate control section 210 is a control section that controls the intermittent communication/disconnection of the electromagnetic valve 203 to intermittently provide, for example, a supply flow rate Vn calculated at the flow rate calculation section 215 to the balloon, and for example, the flow rate control section 210 controls at least one of the communication time of the electromagnetic valve 203 and the supply pressure of the pump 200 to intermittently provide the supply flow rate Vn to the balloon.

It is noted that the flow rate control section 210 is adapted to control the driving of the pump 200 so that it operates at a supply pressure of the maximum capacity, when the internal pressure in the balloon 20 has not reached an initial internal pressure P0, by performing the pressure control of the pump 200 at a sufficiently high gain.

Further, this flow rate control section 210 executes a control, for example, based on the endoscope ON/OFF switch SW4 (see FIG. 1) of the hand switch 104.

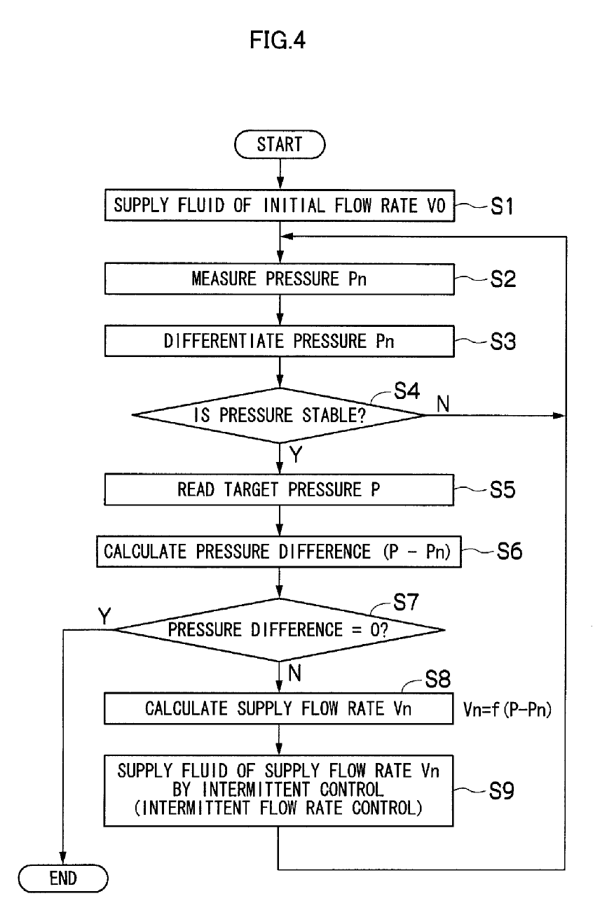

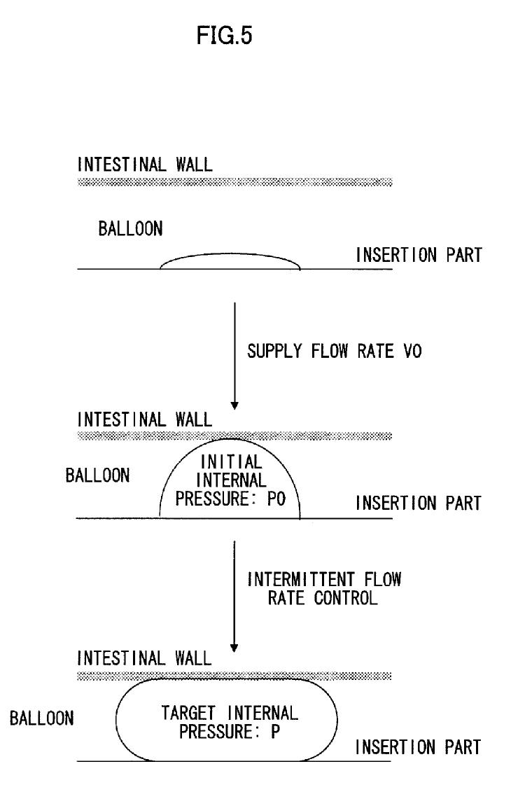

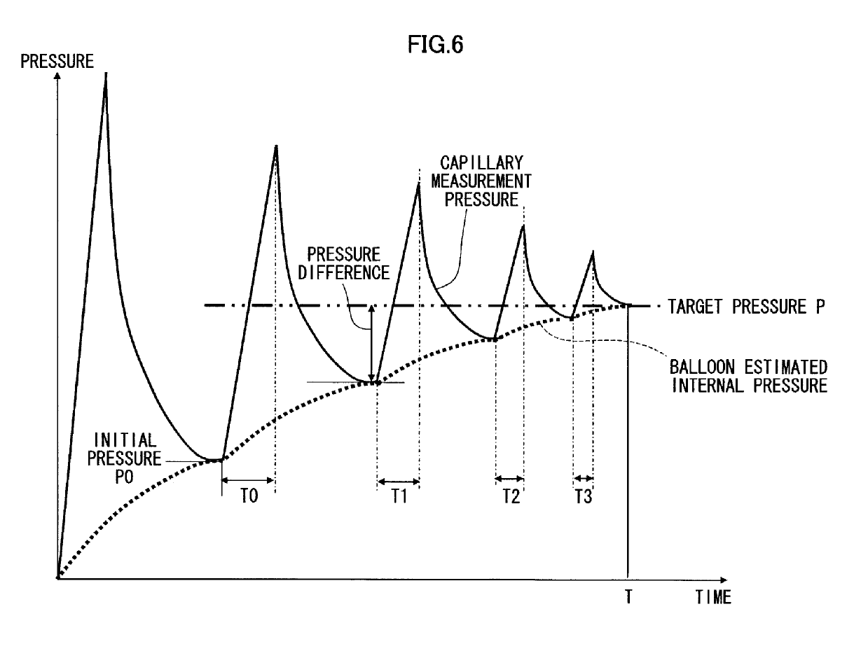

The effect of the balloon control apparatus 100 of the present embodiment configured as described above will be described by using FIGS. 4 to 6. FIG. 4 is a flowchart to show the flow of the control in the balloon control apparatus of FIG. 3, FIG. 5 explains the state of the balloon according to the processing of FIG. 4, and FIG. 6 shows the transition of the internal pressure of the balloon according to the processing of FIG. 4.

The balloon control apparatus 100 performs the control of intermittent communication/disconnection of the electromagnetic valve 203 at the flow rate control section 210 and the pressure control of the pump 200 at a sufficiently high gain to supply air (fluid) of an initial flow rate V0 to the balloon 20 and adjusts the internal pressure of the balloon 20 to be an initial internal pressure P0 which is a safe pressure to be applied to the intestinal wall (step S1).

Next, the balloon control apparatus 100 detects the internal pressure of the tube 110 measured by the pressure gauge 202 at the pressure detection section 211 as a pressure signal (electric signal) (step S2).

Then, the balloon control apparatus 100 differentiates in time the pressure signal at the differentiation circuit 212 (step S3), and determines if the pressure signal is stabilized, by extracting a reached value of the pressure signal at the pressure difference calculation section 213 (step S4).

When it is determined that the pressure signal is stabilized, the balloon control apparatus 100 reads out a target pressure P from the memory 214 at the pressure difference calculation section 213 (step S5). It is noted that if it is determined that the pressure signal is not stabilized, the processing returns to step S2.

Then, the balloon control apparatus 100 calculates a pressure difference (P−Pn) between the internal pressure (pressure signal value) Pn of the tube 110 at a reached value of the pressure signal extracted at the differentiation circuit 212 in the pressure difference calculation section 213, and a target internal pressure P of the balloon 20 which is read out from the memory 214 (step S6).

Next, the balloon control apparatus 100 determines if the pressure difference (P−Pn) is “0” at the pressure difference calculation section 213, that is, if the internal pressure of the balloon 20 has reached the target internal pressure P (step S7). When it is determined that the internal pressure of the balloon 20 has reached the target internal pressure P, the balloon control apparatus 100 ends the processing.

When it is determined that the internal pressure of the balloon 20 has not reached the target internal pressure P, the balloon control apparatus 100 calculates a supply flow rate Vn of air at the time of a next communication of the electromagnetic valve 203 based on a predetermined function f of which variable is the pressure difference (P−Pn) calculated by the pressure difference calculation section 213, at the flow rate calculation section 215 (step S8).

Then, the balloon control apparatus 100 controls the intermittent communication/disconnection of the electromagnetic valve 203 at the flow rate control section 210 to intermittently provide, for example, a supply flow rate Vn calculated at the flow rate calculation section 215 to the balloon (step S9). Specifically, for example, the flow rate control section 210 controls at least one of the communication time of the electromagnetic valve 203 and the supply pressure of the pump 200 to intermittently provide the supply flow rate Vn to the balloon, and thereafter the process returns to step S2.

By the control of the balloon control apparatus 100 shown in FIG. 4, an initial flow rate V0 is provided from when the internal pressure of the balloon 20 is “0” as shown in FIG. 5 (step S1) so that the internal pressure of the balloon 20 changes to an initial internal pressure P0, and further the internal pressure of the balloon 20 reaches the target internal pressure P through the intermittent flow rate control (steps S2 to S9) as shown in FIG. 6.

As described above, in the intermittent flow rate control of the balloon control apparatus 100 of steps S2 to S9, at least one of the communication time of the electromagnetic valve 203 and the supply pressure of the pump 200 is controlled. For example, when an intermittent flow rate control by the communication time of the electromagnetic valve 203 is performed along with the control of the supply pressure of the pump 200, control is performed as shown in FIG. 6 in such a way that the communication time of the electromagnetic valve 203 of the first intermittent flow rate control is “T0”, the communication time of the electromagnetic valve 203 of the second intermittent flow rate control is “T1”, the communication time of the electromagnetic valve 203 of the third intermittent flow rate control is “T2”, and the communication time of the electromagnetic valve 203 of the fourth intermittent flow rate control is “T3”, thus allowing a substantial reduction of the time T required for the internal pressure of the balloon 20 to reach the target internal pressure P.

It is noted that the balloon control apparatus 100 is, although not shown in the figure, able to perform the intermittent flow rate control based on only the communication time with the supply pressure of the pump 200 being kept constant, and is also able to perform the intermittent flow rate control based on only the supply pressure of the pump 200 with the communication time being kept constant. Moreover, the balloon control apparatus 100 is able to perform an intermittent flow rate control by providing, although not shown in the figure, a pressure variable device such as a regulator in the pump 200 thereby controlling the pressure.

Conventionally, to perform a pressure control to inflate/deflate a balloon (inflator body) to a predetermined pressure, control is performed by measuring pressure and simply determining whether air is to be fed further or stopped; however, in the intermittent flow rate control of the present embodiment, since an air feed amount to be fed next is calculated from a measured pressure and an optimum flow rate of air is fed, it becomes possible to substantially reduce the time T required for the internal pressure to reach the target internal pressure P.

So far, the balloon control apparatus of the present invention and the control method thereof have been described in detail; however, the present invention will not be limited to the examples described above, and without saying, various improvements and modifications can be made within a range not departing from the spirit of the present invention.

What is claimed is:

1. A balloon control apparatus, comprising:
an insertion part to be inserted into a luminal part;
a balloon provided at a distal end of the insertion part;
a pipeline connected to the balloon;
a pump that flows a fluid to the balloon via the pipeline;
an intermittent device that causes the pump and the pipeline to be intermittently disconnected/communicated, thereby restricting the flow of the fluid;
a pipeline pressure detection device that detects a pipeline internal pressure of the pipeline;
a flow amount calculation device that calculates a flow amount of the fluid at the time of a next communication of the intermittent device based on the pipeline internal pressure at a reached value of the pipeline internal pressure in a disconnection period of the intermittent device; and
an intermittent operation control device that controls an intermittent operation in the intermittent device based on the flow amount of the fluid calculated by the flow amount calculation device.

2. The balloon control apparatus according to claim 1, wherein
the pipeline pressure detection device detects the pipeline internal pressure of the pipeline between the balloon and the intermittent device.

3. The balloon control apparatus according to claim 1, wherein
the flow amount calculation device includes: a target internal pressure value storage device that prestores a target internal pressure value of the balloon, a reached value detection device that detects a reached value of the pipeline internal pressure in a disconnection period of the intermittent device; and a pressure difference calculation device that calculates a pressure difference between the pipeline internal pressure at the reached value detected by the reached value detection device and the target internal pressure value stored in the target internal pressure value storage device, and calculates a flow amount of the fluid at the time of a next communication of the intermittent device based on the pressure difference calculated by the pressure difference calculation device.

4. The balloon control apparatus according to claim 2, wherein
the flow amount calculation device includes: a target internal pressure value storage device that prestores a target internal pressure value of the balloon, a reached value detection device that detects a reached value of the pipeline internal pressure in a disconnection period of the intermittent device; and a pressure difference calculation device that calculates a pressure difference between the pipeline internal pressure at the reached value detected by the reached value detection device and the target internal pressure value stored in the target internal pressure value storage device, and calculates a flow amount of the fluid at the time of a next communication of the intermittent device based on the pressure difference calculated by the pressure difference calculation device.

5. The balloon control apparatus according to claim 1, wherein
the intermittent operation control device controls at least one of a communication time in the intermittent operation in the intermittent device and a flow pressure of the pump based on the flow amount of the fluid calculated by the flow amount calculation device.

6. The balloon control apparatus according to claim 2, wherein
the intermittent operation control device controls at least one of a communication time in the intermittent operation in the intermittent device and a flow pressure of the pump based on the flow amount of the fluid calculated by the flow amount calculation device.

7. The balloon control apparatus according to claim 3, wherein
the intermittent operation control device controls at least one of a communication time in the intermittent operation in the intermittent device and a flow pressure of the pump based on the flow amount of the fluid calculated by the flow amount calculation device.

8. The balloon control apparatus according to claim 4, wherein
the intermittent operation control device controls at least one of a communication time in the intermittent operation in the intermittent device and a flow pressure of the pump based on the flow amount of the fluid calculated by the flow amount calculation device.

9. The balloon control apparatus according to claim 1, wherein
the pump flows and supplies the fluid to the balloon.

10. The balloon control apparatus according to claim 1, wherein
the pump flows and discharges the fluid from the balloon.

11. The balloon control apparatus according to claim 1, wherein
the insertion part is an insertion part of an endoscope that is inserted into a luminal organ in a body cavity.

12. A method of controlling a balloon control apparatus, comprising the steps of:
an intermittent step of causing a pump that flows a fluid into a balloon provided at a distal end of an insertion part to be inserted into a luminal part, and a pipeline, which is connected to the balloon, to be intermittently disconnected/communicated, thereby restricting the flow of the fluid;
a pipeline pressure detection step of detecting a pipeline internal pressure of the pipeline;
a flow amount calculation step of calculating a flow amount of the fluid at the time of a next communication of the intermittent step based on the pipeline internal pressure at a reached value of the pipeline internal pressure in a disconnection period of the intermittent step; and
an intermittent operation control step of controlling an intermittent operation in the intermittent step based on the flow amount of the fluid calculated by the flow amount calculation step.
